(19) United States
(12) Patent Application Publication
(10) Pub. No.: US 2005/0159818 A1
(43) Pub. Date: Jul. 21, 2005

(54) Total disc replacement system and related methods

(76) Inventor: Jason Blain, San Diego, CA (US)

Correspondence Address: Nuvasive, Inc.;c/o PortfolioIP, P.O. Box 52050, Minneapolis, MN, 55402, US

(21) Appl. No.: 11/016,044

(22) Filed: Dec. 17, 2004

Related U.S. Application Data
(63) Continuation of application No. PCT/US03/20333, filed on Jun. 26, 2003.

(51) Int. Cl. A61F 2/44

(52) U.S. Cl. 623/17.15; 623/17.14

(57) ABSTRACT

A total disc replacement (TDR) system for use in the spine and related methods, involving a first anchor plate having a first surface for engaging a first vertebra and a second surface including a semi-cylindrical articular surface, a second anchor plate having a first surface for engaging a second vertebra and a second surface including a semi-cylindrical articular surface, and an intradiscal element including a first articular surface having a generally arcuate cross-section for articulating with said semi-cylindrical articular surface of said first anchor plate, and a second articular surface having a generally arcuate cross-section for articulating with said semi-cylindrical articular surface of said second anchor plate.

### CROSS-REFERENCES TO RELATED APPLICATIONS

This application is a continuation under 35 U.S.C. 111(a) of PCT Patent Application Serial No. PCT/U.S. 2003/20333, filed Jun. 26, 2003 and published on Jan. 8, 2004 as WO 2004/002291 A2 which is incorporated herein by reference.

The present application also claims the benefit of priority from U.S. Provisional Patent Application Ser. No. 60/392,206 (filed Jun. 26, 2002), the entire content of which is hereby expressly incorporated by reference into this disclosure as if set forth fully herein.

### BACKGROUND OF THE INVENTION

I. Field of the Invention

The present invention relates generally to spinal surgery and, more particularly, to total disc replacement systems and related methods.

II. Description of Related Art

In recent years, the area of total disc replacement has experienced proliferated growth and attention from the medical community. Known total disc replacement devices generally require some form of articulation or inherent flexibility in the device to permit a spine having the device to maintain its natural posture and range of motion as much as possible. Such devices typically include between 2 and 4 separate components constructed from any number of materials, such as plastic, rubber, metal, ceramic and alloys. Generally speaking, these components include a pair of anchor plates for engagement with opposed vertebral body endplates and one or more internal components for simulating the intervertebral disc.

Known total disc replacement systems suffer disadvantages including the dislocation of the anchor plates from the vertebral end plates, over-distraction of the vertebral endplates during introduction, particulate wear and debris of the component parts, and a lack of conformity between the anchor plates and the internal components during use. The present invention is directed at overcoming, or at least reducing the effects of, one or more of the problems set forth above.

### SUMMARY OF THE INVENTION

The present invention solves the above-identified drawbacks with the prior art by providing a total disc replacement system including a pair of anchor plates and an intradiscal element. The first anchor plate has a first surface for engaging a first vertebra and a second surface including a semi-cylindrical articular surface. The second anchor plate has a first surface for engaging a second vertebra and a second surface including a semi-cylindrical articular surface. The intradiscal element includes a first articular surface having a generally arcuate cross-section for articulating with the semi-cylindrical articular surface of the first anchor plate, and a second articular surface having a generally arcuate cross-section for articulating with the semi-cylindrical articular surface of the second anchor plate. As used herein, the term “semi-cylindrical” is defined to mean any cylindrical shape which has a cross-section of less than a full circle.

The first articular surface is dimensioned to articulate with the semi-cylindrical articular surface of the first anchor plate such that the first anchor plate may rotate relative to the intradiscal element about a first axis (e.g. Z-axis), as well as translate relative to the intradiscal element in either direction along the first axis. The second articular surface is dimensioned to articulate with the semi-cylindrical articular surface of the second anchor plate such that the second anchor plate may rotate relative to the intradiscal element about a second axis (e.g., X-axis), as well as translate relative to the intradiscal element in either direction along the second axis. In this fashion, rotation about the first axis will always occur at the same location along the first anchor plate and rotation about the second axis will always occur at the same location along the second anchor plate. In an optional configuration, the intradiscal element may (by way of example only) comprise a first intradiscal element rotatably coupled to a second intradiscal element, which allows relative rotation along a third axis (e.g., Y-axis). In use, then, the TDR system of the present invention provides rotation along three distinct axes (e.g., X, Y, Z) and translation along two distinct axes (e.g., X and Z).

The semi-cylindrical articular surface of the first and/or second anchor plates may be generally concave and/or generally convex. The generally arcuate cross-sections of the first and/or second articular surfaces of the intradiscal element may be generally concave and/or convex. The second surface of the first and/or second anchor plates may include a ramped portion to facilitate the introduction of the intradiscal element into articulating engagement with the semi-cylindrical articular surfaces of the first and second anchor plates. The intradiscal element may be prevented from translating relative to the first and/or second anchor plates.

The first and second anchor plates may each include an anchor element for anchoring into adjacent vertebrae. The anchor elements may include a plurality of protrusions. In one aspect, the anchor elements are oriented such that the first and second anchor plates may be introduced in a generally lateral approach relative to the first and second vertebrae. In another aspect, the anchor elements are oriented such that the first and second anchor plates may be introduced in a generally anterior approach relative to the first and second vertebrae.

The intradiscal element may take any number of different forms. In one aspect, the intradiscal element is generally spherical and may be provided as a single generally spherical member or at least two semi-spherical members coupled together. In another aspect, intradiscal element may include at least one generally semi-cylindrical articular surface for articulating with the semi-cylindrical articular surface of the first and/or second anchor plate. In this embodiment, the intradiscal element may be provided as a unitary member and/or may comprise a first intradiscal element rotatably coupled to a second intradiscal element via (by way of example only) a post.

The first anchor plate, second anchor plate, and/or intradiscal element may be provided with at least one lumen for engagement with an insertion tool.

The first anchor plate, second anchor plate, and/or intradiscal element may be constructed from metal, ceramic, polymer, and/or any combination thereof.

According to a further embodiment of the present invention, a disc replacement system is provided for lateral introduction into the spine comprising a pair of anchor plates and an intradiscal element. The first anchor plate has a first surface for engaging a first vertebra and a second surface including a semi-cylindrical articular surface. The first anchor plate has a generally rectangular shape including a length in the range of 15 mm and 40 mm and a width of less than 25 mm. The second anchor plate has a first surface for engaging a second vertebra and a second surface including a semi-cylindrical articular surface. The second anchor plate has a generally rectangular shape including a length in the range of 15 mm and 40 mm and a width of less than 25 mm. The intradiscal element includes a first articular surface having a generally arcuate cross-section for articulating with the semi-cylindrical articular surface of the first anchor plate, and a second articular surface having a generally arcuate cross-section for articulating with the semi-cylindrical articular surface of the second anchor plate.

### BRIEF DESCRIPTION OF THE DRAWINGS

Other objects and advantages of the invention will become apparent upon reading the following detailed description and upon reference to the drawings in which:

FIG. 1 is a perspective view of a total disc replacement system according to a first embodiment of the present invention;

FIG. 2 is an exploded perspective view of the total disc replacement system according to the first embodiment of the present invention;

FIG. 3 is a front view of the total disc replacement system according to the first embodiment of the present invention (e.g. anterior view of TDR in use);

FIG. 4 is a rear view of the total disc replacement system according to the first embodiment of the present invention (e.g. posterior view of TDR in use);

FIG. 5 is a side view of the total disc replacement system according to the first embodiment of the present invention (e.g. lateral view of TDR in use);

FIGS. 6-9 are perspective, front, top, and side views, respectively, of a first anchor plate forming part of the total disc replacement system according to the first embodiment of the present invention;

FIGS. 10-13 are perspective, front, top, and side views, respectively, of a second anchor plate forming part of the total disc replacement system according to the first embodiment of the present invention;

FIG. 14 is a perspective view of an intradiscal element forming part of the total disc replacement system according to an embodiment of the present invention;

FIG. 15 is an exploded perspective view of an intradiscal element forming part of the total disc replacement system according to an embodiment of the present invention;

FIGS. 16-19 are perspective, front, side, and bottom views, respectively, of a first intradiscal element according to an embodiment of the present invention;

FIGS. 20-23 are perspective, side, top, and end views, respectively, of a second intradiscal element according to an embodiment of the present invention;

FIG. 24 is a perspective view of a total disc replacement system according to a second embodiment of the present invention;

FIG. 25 is an exploded perspective view of the total disc replacement system according to the second embodiment of the present invention;

FIGS. 26-27 are front and back views of the total disc replacement system according to the second embodiment of the present invention (e.g. anterior and posterior views of TDR in use);

FIG. 28 is a side view of the total disc replacement system according to the second embodiment of the present invention (e.g. lateral view of TDR in use);

FIGS. 29-32 are perspective, front, top, and side views, respectively, of a first anchor plate forming part of the total disc replacement system according to the second embodiment of the present invention; and

FIGS. 33-36 are perspective, front, top, and side views, respectively, of a second anchor plate forming part of the total disc replacement system according to the second embodiment of the present invention.

### DESCRIPTION OF THE SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The systems disclosed herein boast a variety of inventive features and components that warrant patent protection, both individually and in combination.

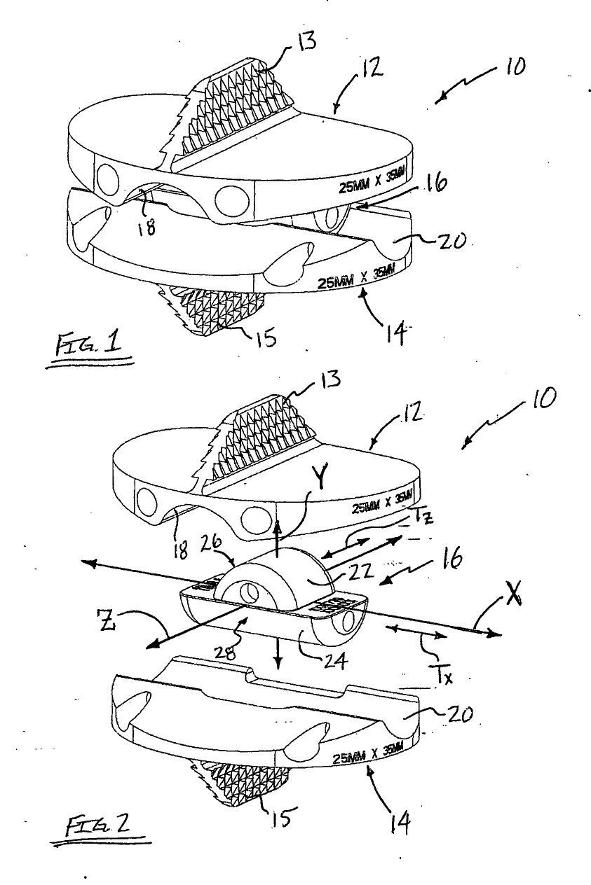

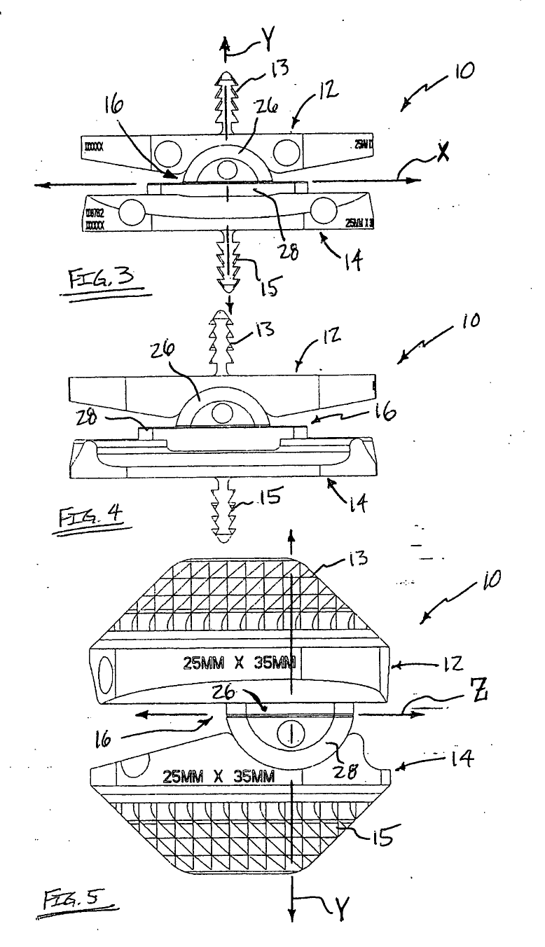

FIGS. 1-5 illustrate a total disc replacement (TDR) system 10 according to a first embodiment of the present invention. The TDR system 10 includes a first anchor plate 12, a second anchor plate 14, and an intradiscal element 16. Each anchor plate 12 and 14 is equipped with an anchor element 13, 15 and a semi-cylindrical articular surface 18, 20, respectively. The intradiscal element 16 includes a first articular surface 22 and a second articular surface 24, each having a generally arcuate cross-section. The first articular surface 22 is dimensioned to articulate with the semi-cylindrical articular surface 18 of the first anchor plate 12 such that the first anchor plate 12 may rotate relative to the intradiscal element 16 about the Z-axis, as well as translate relative to the intradiscal element 16 in either direction along the Z-axis (denoted as line “T_{Z}”). The second articular surface 24 is dimensioned to articulate with the semi-cylindrical articular surface 20 of the second anchor plate 14 such that the second anchor plate 14 may rotate relative to the intradiscal element 16 about the X-axis, as well as translate relative to the intradiscal element 16 in either direction along the X-axis (denoted as line “T_{X}”). In this fashion, rotation about the Z-axis will always occur at the same location along the first anchor plate 12 and rotation about the X-axis will always occur at the same location along the second anchor plate 14. In an optional configuration, the intradiscal element 16 may (by way of example only) comprise a first intradiscal element 26 rotatably coupled to a second intradiscal element 28, which allows relative rotation along the Y-axis. In use, then, the TDR system 10 of this first embodiment provides rotation along three distinct axes (X, Y, Z) and translation along two distinct axes (X and Z).

When used within the lumbar spine, for example, it may be desirable to configure the second anchor plate 14 such that the semi-cylindrical articular surface 20 (and hence X-axis) is located within the posterior one-third of the disc space (and generally within the frontal plane of the patient) to approximate the axis of rotation of the natural spine during flexion and extension. It may similarly be desirable to configure the first anchor plate 12 such that the semi-cylindrical articular surface 18 (and hence Z-axis) is located at the approximate center of the disc space (and generally within the sagital plane of the patient) to approximate the axis of rotation of the natural spine during lateral bending. Although described by way of example in this configuration, it will be appreciated that the relative position of the semi-cylindrical articular surfaces 18, 20 may be altered in any number of different fashions depending upon the vertebral level (i.e. cervical, thoracic, and/or lumbar) as well as the directional approach employed to place the TDR system 10 into a disc space (e.g., lateral, anterior, postero-lateral, antero-lateral). Moreover, it will be appreciated that the TDR system 10 may be introduced into a disc space in the orientation shown (with the first anchor plate 12 “above” the second anchor plate 14 such that the anchor elements 13, 15 are to be disposed within a respective “upper” and “lower” vertebral level within the patient) or vice versa.

FIGS. 6-9 illustrate the first anchor plate 12 in greater detail. First anchor plate 12 includes a generally planar surface 30 for engaging against a vertebra, and a pair of generally angled surfaces 32 extending in a ramp-like fashion away from the semi-cylindrical articular surface 18 towards the lateral edges of the first anchor plate 12. The angled surfaces 32 serve to limit the relative rotation about the Z-axis. That is, the first anchor plate 12 will be able to rotate about the Z-axis until an angled surface 32 comes into contact with another structure, such as the second intradiscal element 28 or the second anchor plate 14 (e.g., if the second intradiscal element 28 is shorter or if the intradiscal element 16 is spherical). As will be described in greater detail below, the second intradiscal element 28 may have a generally flat or angled surface against which the angled surfaces 32 may abut to accomplish the desired rotational limitation in the Z-axis.

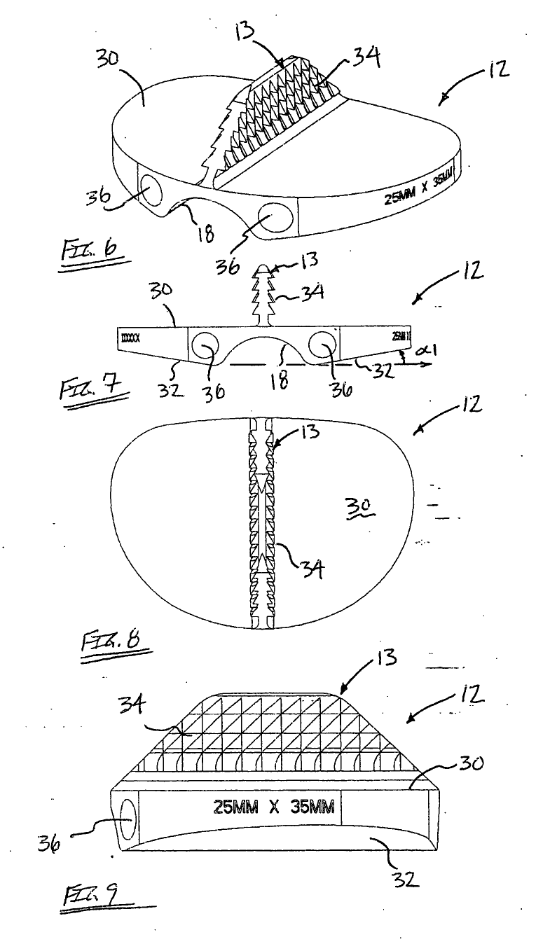

In a preferred embodiment, the angled surfaces 32 may be provided in the range ∂1 of between 0 and 20 degrees each way from the generally horizontal position shown best in FIG. 7. If the TDR system 10 is provided for placement via an anterior approach (shown by way of example), this would allow for lateral bending in the range of between 0 and 20 degrees in either direction. In this instance, the anchor element 13 would be introduced into a first vertebra in the sagital plane.

A plurality of anti-migration features 34 may be provided on the anchor element 13 to inhibit the movement of the anchor element 13 after introduction into a slot or receiving area formed within a vertebra. In one embodiment, the anti-migration features 34 comprise teeth having a generally triangular cross-section, although any number of suitable configurations or anti-migration elements may be employed without departing from the scope of the present invention. Any number of mechanisms or techniques may be employed to introduce the first anchor plate 12 into a vertebra, including but not limited to providing one or more lumens 36 in the first anchor plate for coupling to or engaging with an insertion tool (not shown).

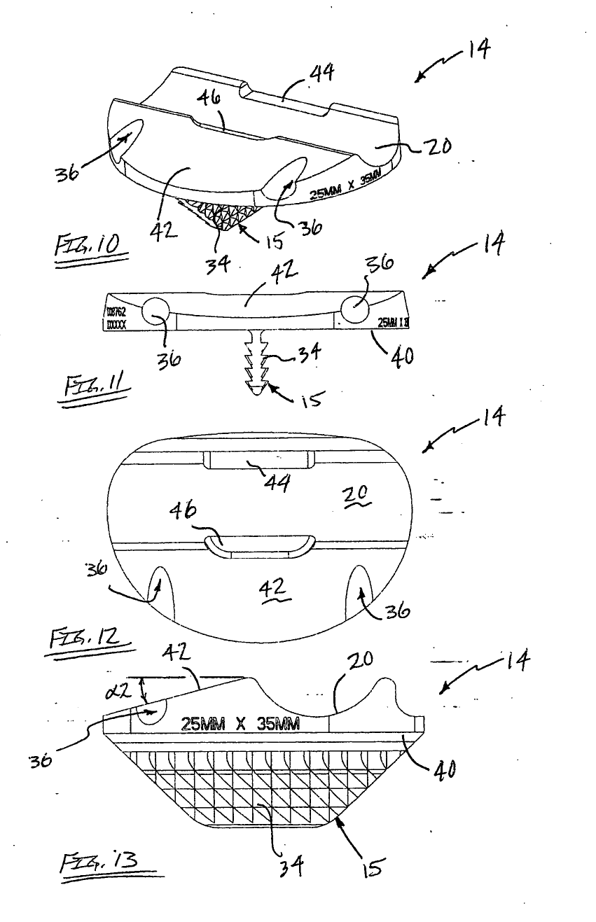

FIGS. 10-13 illustrate the second anchor plate 14 in greater detail. Second anchor plate 14 includes a generally planar surface 40 for engaging against a vertebra, a generally angled surface 42, and a pair of recessed regions 44, 46. The recessed regions 44, 46 are provided at the approximate mid-line or middle of the semi-cylindrical articular surface 20 and are generally width, long, and deep enough to permit the first intradiscal element 26 to rotate relative to the second intradiscal element 28 without contacting the second anchor plate 14, particularly during translation of the intradiscal element 16 along the X-axis.

The generally angled surface 42 extends in a ramp-like fashion away from the semi-cylindrical articular surface 20 towards the anterior edge of the second anchor Plate 14. The angled surface 42 serves two functions. The first function is to limit the relative rotation along the X-axis. That is, the second anchor plate 14 will be able to rotate about the X-axis until the angled surface 42 comes into contact with the first anchor plate 12. The second function is to facilitate the introduction of the intradiscal element 16. That is, if the first and second anchor plates 12, 14 are introduced first, then the angled surface 42 will serve as a ramp over which the intradiscal element 16 may pass in order to become seated in an articulating relationship with the semi-cylindrical articular surfaces 18, 20. In this fashion, the ramp effect of the angled surface 42 will cause the first and second anchor plates 12, 14 (and their respective vertebrae) to gently distract to receive the intradiscal element 16.

In a preferred embodiment, the angled surface 42 may be provided in the range ∂2 of between 1 and 20 degrees in either direction from the horizontal as shown in FIG. 12. If the TDR system 10 is provided for placement via an anterior approach (shown by way of example), this would allow for flexion in the range of between 1 and 20 degrees. Extension would be limited in this example by the degree to which the first anchor plate 12 could rotate about the X-axis before coming into contact with the portion of the second anchor plate 14 on either side of the recessed region 44. In this instance, the anchor element 15 would be introduced into a second vertebra in the sagital plane. The anchor element 15 may be equipped with the same anti-migration features 34 discussed above with reference to anchor element 13 such that a repeat discussion is not necessary. In similar fashion described above with reference to the first anchor plate 12, any number of mechanisms or techniques may be employed to introduce the second anchor plate 14 into a vertebra, including but not limited to providing one or more lumens 36 in the first anchor plate for coupling to or engaging with an insertion tool (not shown).

The first and second anchor plates 12, 14 of the present invention may be constructed from any number of materials and/or compositions suitable for medical applications, including but not limited to metallic compositions or alloys (such as Co—Cr—Mo), ceramics (such as zirconia and/or alumina), polymers (such as ultra-high molecular weight polyethylene), and/or any combination thereof. Where beneficial and appropriate, either or both of the first and second anchor plates 12, 14 may also be coated with any number of suitable compositions, such as zirconium oxide coating found in U.S. Pat. No. 5,037,438, the contents of which are hereby incorporated into this disclosure as if set forth in its entirety.

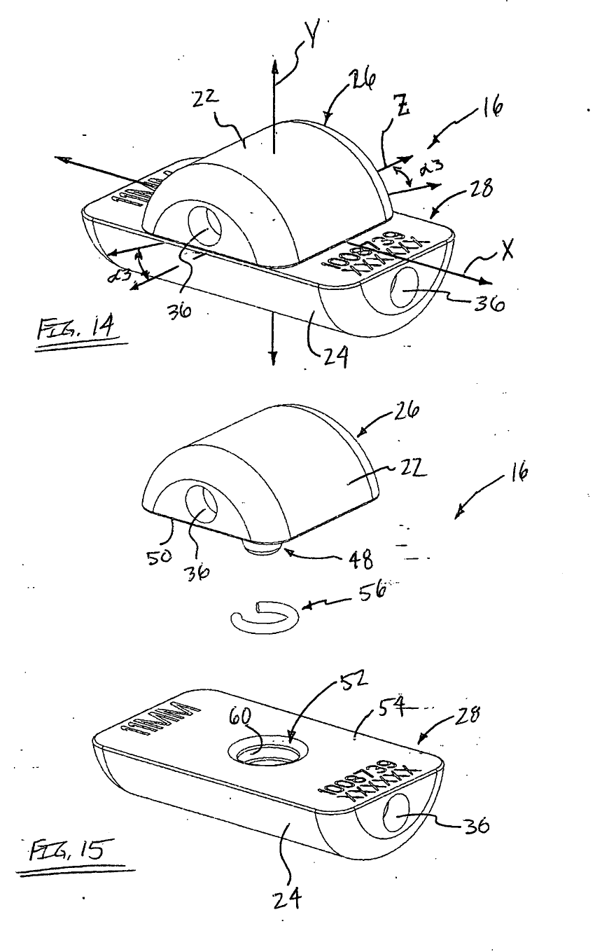

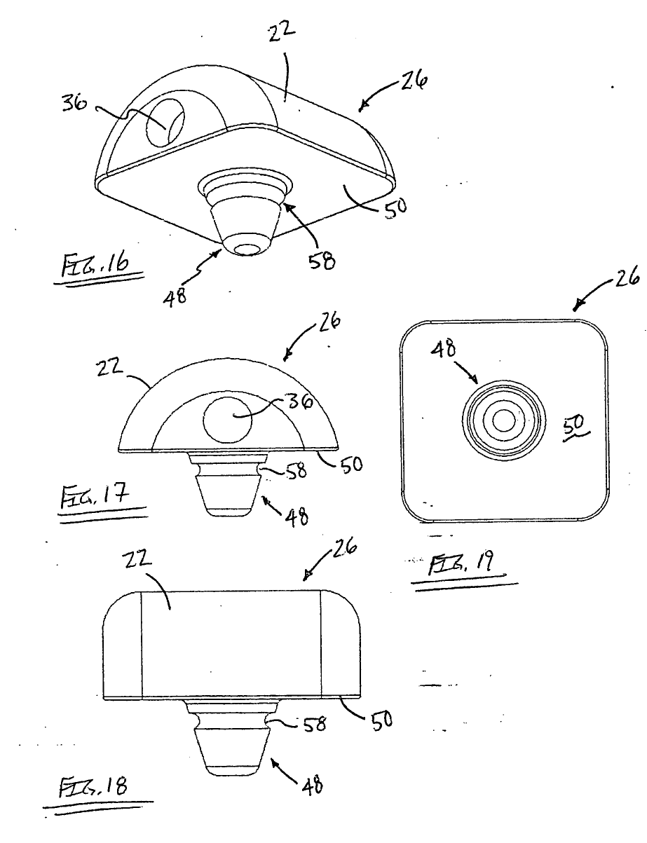

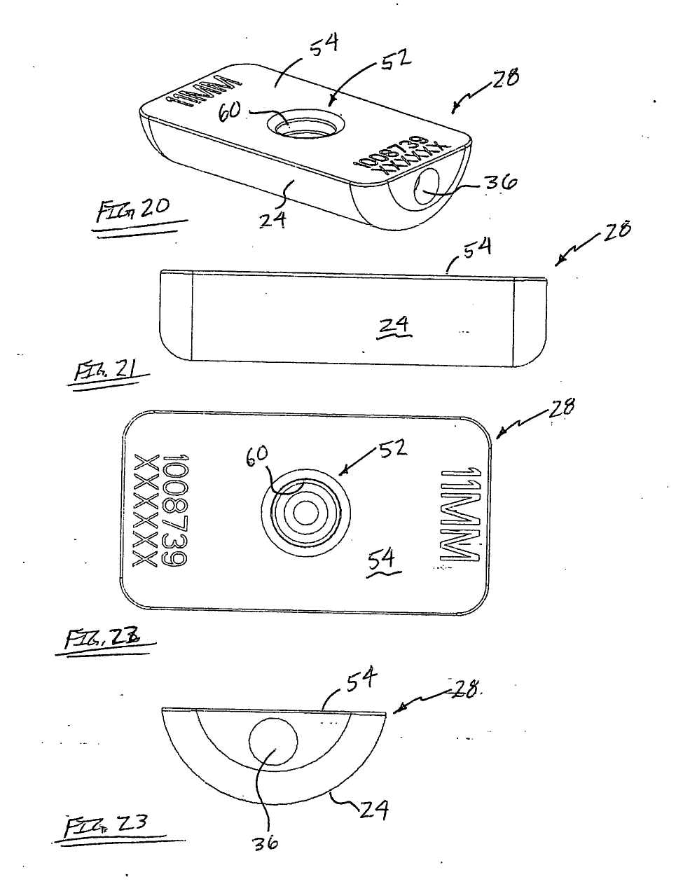

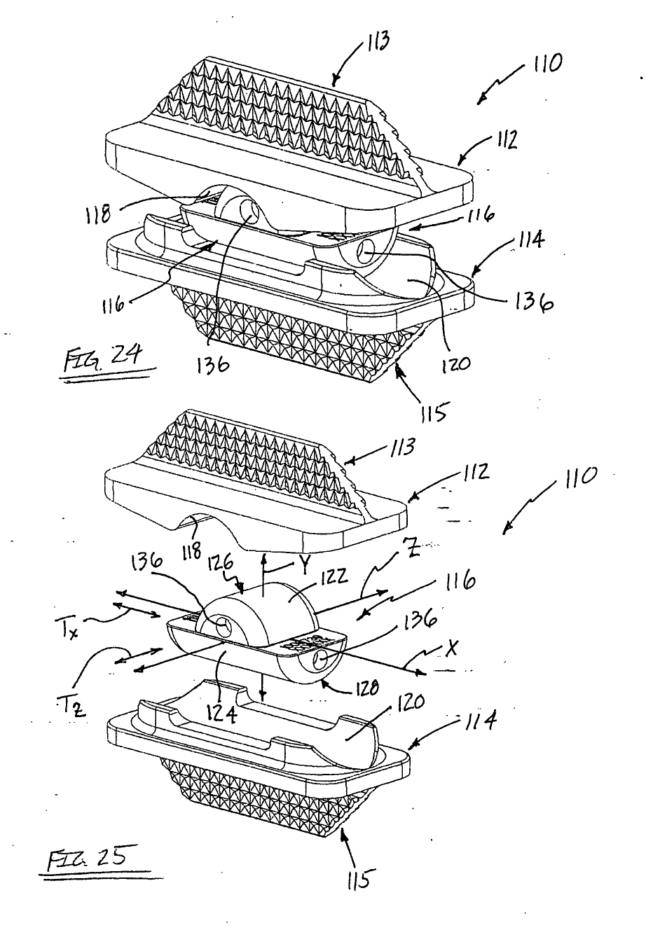

FIGS. 14-15 illustrate the intradiscal element 16 in greater detail. In this embodiment, the intradiscal element 16 includes the first intradiscal element 26 rotatably coupled to the second intradiscal element 28. By way of example only, this rotational relationship is accomplished by providing a post element 48, an aperture 52, and a snap-ring 56. As best shown in FIGS. 16-19, the post element 48 extends from a generally planar surface 50 of the first intradiscal element 26, and preferably from the approximate center thereof. The post element 48 includes a groove 58 dimensioned to receive the snap-ring 56 in a biased configuration therein. As best shown in FIGS. 20-23, the aperture 52 is formed in a generally planar surface 54 of the second intradiscal element 28, and the optional snap-ring 56 is dimensioned to be engaged within a groove 58 formed along the post element 48 and a groove 60 formed within the aperture 52.

FIG. 14 best illustrates the aspect of rotation between the first and second intradiscal elements 26, 28. The first and second intradiscal elements 26, 28 may be initially oriented in any number of different manners, including but not limited to the generally perpendicular orientation shown. From this position, the first and second intradiscal elements 26, 28 may be rotated about the Y-axis in the range ∂3 of between 0 and 20 degrees in either direction from the Z-axis shown in FIG. 14 (within the plane of the generally planar surfaces 50, 54 of the first and second intradiscal elements 26, 28). If the TDR system 10 is provided for placement via an anterior approach (shown by way of example), this would allow the first anchor plate 12 to rotate relative to the second anchor plate 14 in the range of between 0 and 20 degrees in either direction about the Y-axis.

In addition to the rotational capability about the Y-axis, the first articular surface 22 is dimensioned to articulate with the semi-cylindrical articular surface 18 of the first anchor plate 12, while the second articular surface 24 is dimensioned to articulate with the semi-cylindrical articular surface 20 of the second anchor plate 14. This enables the first anchor plate 12 to rotate relative to the intradiscal element 16 about the Z-axis, and the second anchor plate 14 may rotate relative to the intradiscal element 16 about the X-axis. As noted above, this also enables the first anchor plate 12 to translate relative to the intradiscal element 16 in either direction along the Z-axis, and the second anchor plate 14 to translate relative to the intradiscal element 16 in either direction along the X-axis. In this fashion, the TDR system 10 of this first embodiment provides rotation along three distinct axes (X, Y, Z) and translation along two distinct axes (X and Z).

The intradiscal element 16 of the present invention may be constructed from any number of materials and/or compositions suitable for medical applications, including but not limited to metallic compositions or alloys (such as Co—Cr—Mo), ceramics (such as zirconia and/or alumina), polymers (such as ultra-high molecular weight polyethylene), and/or any combination thereof. Where beneficial and appropriate, the intradiscal element 16 may also be coated with any number of suitable compositions, such as zirconium oxide coating found in U.S. Pat. No. 5,037,438, the contents of which are hereby incorporated into this disclosure as if set forth in its entirety.

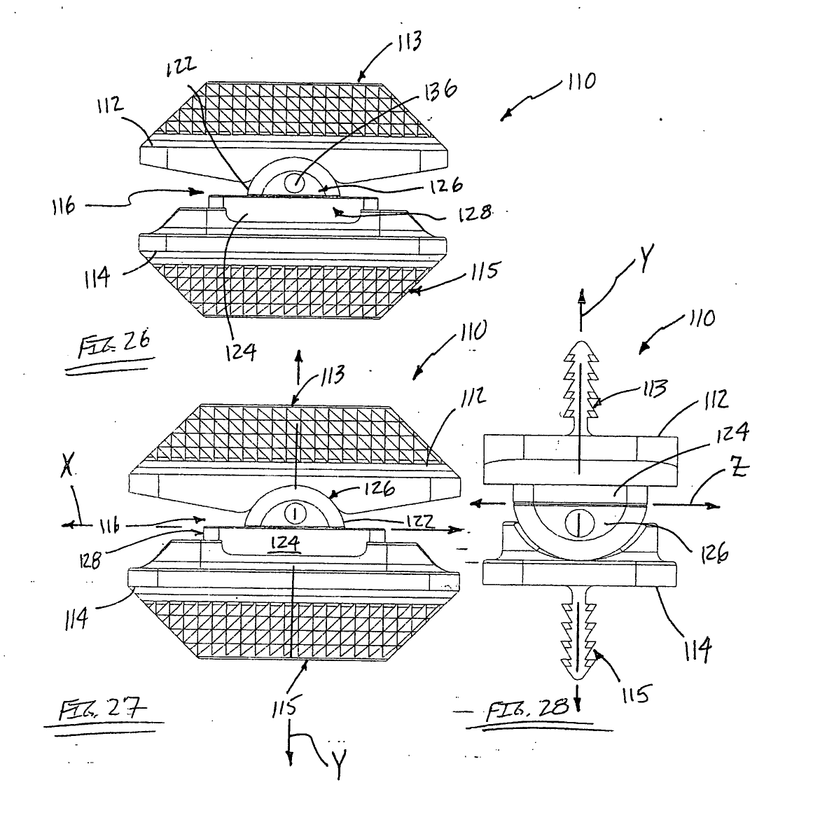

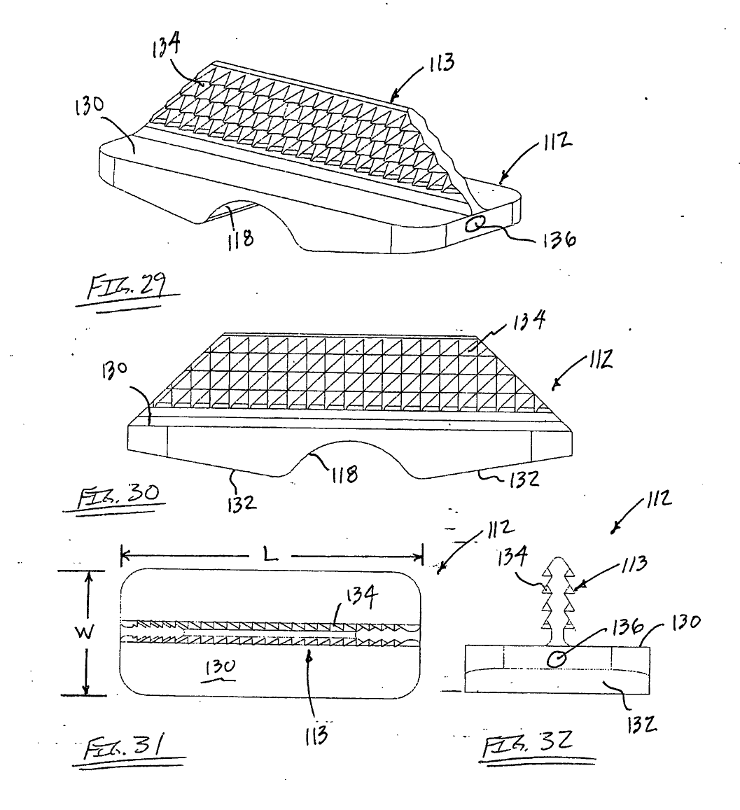

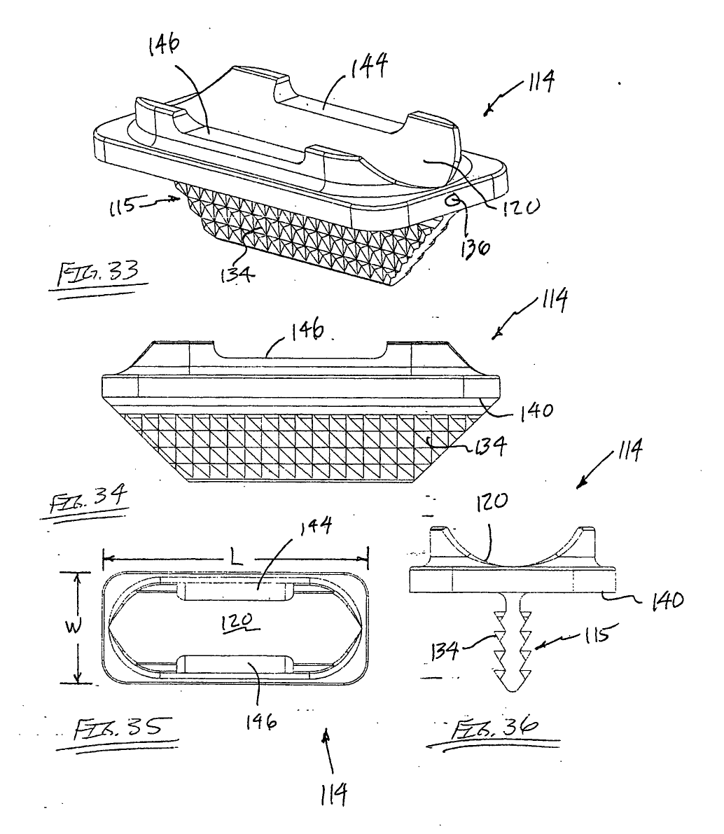

FIGS. 24-36 illustrate a total disc replacement (TDR) system 110 according to a second embodiment of the present invention. The TDR system 110 is designed to operate in the same fashion as the TDR system 10 shown and described above, except that the TDR system 110 is particularly suited for lateral and/or minimal access introduction into the spine. (For the sake of clarity, all features or components in common with the TDR system 10 will be numbered “+100” and, as to those features, reference can be made to the discussion above regarding the TDR system 10, rendering a repeat discussion unnecessary and optional). To accomplish this, as best viewed respectively in FIGS. 31 and 35, the first and second anchor plates 112, 114 each have a generally rectangular shape including a width of less than 25 mm. Dimensioning the first and second anchor plates 112, 114 in this fashion advantageously provides the ability to advance the TDR system 110 through a minimal access surgical corridor. It also advantageously provides the ability to introduce the TDR system 110 into the spine using a generally lateral approach. The lateral approach capability is augmented by orienting the anchor element 113, 115 approximately ninety degrees relative to that of the TDR system 10, such that the anchor elements 113, 115 are introduced into the respective vertebra such that they are disposed generally within the frontal plane of the patient. Depending upon the particular patient, and the location within the particular vertebral level within that patient, the anchor plates 112, 114 may be provided with a length in the range of between 15 mm and 40 mm.

The first articular surface 122 is dimensioned to articulate with the semi-cylindrical articular surface 118 of the first anchor plate 112 such that the first anchor plate 112 may rotate relative to the intradiscal element 116 about the Z-axis, as well as translate relative to the intradiscal element 116 in either direction along the Z-axis. The second articular surface 124 is dimensioned to articulate with the semi-cylindrical articular surface 120 of the second anchor plate 114 such that the second anchor plate 114 may rotate relative to the intradiscal element 116 about the X-axis, as well as translate relative to the intradiscal element 116 in either direction along the X-axis. In this fashion, rotation about the Z-axis will always occur at the same location along the first anchor plate 112 and rotation about the X-axis will always occur at the same location along the second anchor plate 114. In an optional configuration, the intradiscal element 116 may (by way of example only) comprise the intradiscal element 16 shown and described above. In use, then, the TDR system 110 of this first embodiment provides rotation along three distinct axes (X, Y, Z) and translation along two distinct axes (X and Z).

The TDR systems of the present invention may be provided with various modifications without departing from the scope of the invention. For example, the semi-cylindrical articular surfaces of the first and/or second anchor plates may be generally convex in addition to the generally concave configuration shown. In similar fashion, the generally arcuate cross-sections of the first and/or second articular surfaces of the intradiscal element may be generally concave in addition to the generally convex configuration shown. Moreover, the intradiscal element may be prevented from translating relative to the first and/or second anchor plates in any suitable fashion, such as by equipping the either or both of the anchor plates and/or the intradiscal element with a structure (e.g. a wall member extending from the anchor plate) or a stop along the length of the first and/or second semi-cylindrical articular surfaces.

It should be noted with particularity that the intradiscal element 16 may take any number of forms without departing from the scope of the invention, provided it has a first articular surface and a second articular surface each having a generally arcuate cross-section. That is, in addition to the “dual mating semi-cylinders” configuration described above (with the first generally semi-cylindrical intradiscal element 26 rotatably coupled to the second generally semi-cylindrical intradiscal element 28), the intradiscal element 16 may be provided as a generally spherical configuration (of unitary or multi-component construction with the constituent components fused together or rotatable relative to one another), a “dual fixed semi-cylinder” configuration (wherein the first and second intradiscal elements 26, 28 are provided as a single element of unitary construction or multi-component construction with the constituent components fused or otherwise locked together), or any combination of the foregoing (e.g. with a semi-cylinder portion rotatably or fixedly coupled to a dome-shaped portion). In each instance, the language “first articular surface and a second articular surface, with each having a generally arcuate cross-section” shall include, but not necessarily be limited to, any or all of these variations.

While this invention has been described in terms of a best mode for achieving this invention's objectives, it will be appreciated by those skilled in the art that variations may be accomplished in view of these teachings without deviating from the spirit or scope of the present invention. As can be envisioned by one of skill in the art, many different combinations of the above may be used and accordingly the present invention is not limited by the scope of the appended claims.

1. A disc replacement system for use in the spine, comprising:
a first anchor plate having a first surface for engaging a first vertebra and a second surface including a semi-cylindrical articular surface;
a second anchor plate having a first surface for engaging a second vertebra and a second surface including a semi-cylindrical articular surface; and
an intradiscal element including a first articular surface having a generally arcuate cross-section for articulating with said semi-cylindrical articular surface of said first anchor plate, and a second articular surface having a generally arcuate cross-section for articulating with said semi-cylindrical articular surface of said second anchor plate.

2. The disc replacement system of claim 1 and further, wherein said semi-cylindrical articular surface of at least one of said first and second anchor plates is generally concave.

3. The disc replacement system of claim 1 and further, wherein said semi-cylindrical articular surface of at least one of said first and second anchor plates is generally convex.

4. The disc replacement system of claim 1 and further, wherein at least one of said generally arcuate cross-sections of said first and second articular surfaces of said intradiscal element is generally concave.

5. The disc replacement system of claim 1 and further, wherein at least one of said generally arcuate cross-sections of said first and second articular surfaces of said intradiscal element is generally convex.

6. The disc replacement system of claim 1 and further, wherein at least one of second surfaces of said first and second anchor plates includes a ramped portion to facilitate the introduction of said intradiscal element into articulating engagement with said semi-cylindrical articular surfaces of said first and second anchor plates.

7. The disc replacement system of claim 1 and further, wherein said intradiscal element is prevented from translating relative to one of said first and second anchor plates.

8. The disc replacement system of claim 1 and further, wherein said first anchor plate includes an anchor element extending from said first surface for anchoring into said first vertebra and said second anchor plate includes an anchor element extending from said second surface for anchoring into said second vertebra.

9. The disc replacement system of claim 8 and further, wherein at least one of said anchor elements includes a plurality of protrusions.

10. The disc replacement system of claim 8 and further, wherein said anchor elements are oriented such that said first and second anchor plates may be introduced in a generally lateral approach relative to the first and second vertebrae.

11. The disc replacement system of claim 8 and further, wherein said anchor elements are oriented such that said first and second anchor plates may be introduced in a generally anterior approach relative to the first and second vertebrae.

12. The disc replacement system of claim 1 and further, wherein said intradiscal element is generally spherical.

13. The disc replacement system of claim 12 and further, wherein said intradiscal element comprises at least one of a single generally spherical member and at least two semi-spherical members coupled together.

14. The disc replacement system of claim 1 and further, wherein said intradiscal element includes at least one generally semi-cylindrical articular surface for articulating with at least one of said semi-cylindrical articular surface of said first anchor plate and said second anchor plate.

15. The disc replacement system of claim 14 and further, wherein said intradiscal element is a unitary member.

16. The disc replacement system of claim 14 and further, wherein said intradiscal element comprises a first intradiscal element rotatably coupled to a second intradiscal element.

17. The disc replacement system of claim 16 and further, wherein said first intradiscal element is rotatably coupled to said second intradiscal element via a post.

18. The disc replacement system of claim 1 and further, wherein at least one of said first anchor plate, said second anchor plate, and said intradiscal element includes at least one lumen for engagement with an insertion tool.

19. The disc replacement system of claim 1 and further, wherein at least one of said first anchor plate, said second anchor plate, and said intradiscal element is constructed from at least one metal, ceramic, polymer, and a combination thereof.

20. A disc replacement system for lateral introduction into the spine, comprising:
a first anchor plate having a first surface for engaging a first vertebra and a second surface including a semi-cylindrical articular surface, said first anchor plate having a generally rectangular shape including a length in the range of 15 mm and 40 mm and a width of less than 25 mm;
a second anchor plate having a first surface for engaging a second vertebra and a second surface including a semi-cylindrical articular surface, said second anchor plate having a generally rectangular shape including a length in the range of 15 mm and 40 mm and a width of less than 25 mm; and
an intradiscal element including a first articular surface having a generally arcuate cross-section for articulating with said semi-cylindrical articular surface of said first anchor plate, and a second articular surface having a generally arcuate cross-section for articulating with said semi-cylindrical articular surface of said second anchor plate.
